(19) United States
(12) Patent Application Publication
(10) Pub. No.: US 2025/0160308 A1
(43) Pub. Date: May 22, 2025

(54) NUCLEIC ACID CONSTRUCT BASED ON CRE-LOXP AND CRISPR AND USE THEREOF

(71) Applicant: SHANGHAITECH UNIVERSITY, Shanghai (CN)

(72) Inventors: Tian CHI, Shanghai (CN); Bo LIU, Shanghai (CN); Zhengyu JING, Shanghai (CN); Xiaoming ZHANG, Shanghai (CN); Shaoshuai MAO, Shanghai (CN); Yuxin CHEN, Shanghai (CN)

(21) Appl. No.: 18/840,525

(22) PCT Filed: May 17, 2023
(86) PCT No.: PCT/CN2023/094885

§ 371 Date: Aug. 22, 2024

(30) Foreign Application Priority Data
May 27, 2022 (CN) 202210594619.4

(51) Int. Cl. A01K 67/0278; C12N 9/12; C12N 9/22; C12N 15/11; C12N 15/90

(52) U.S. Cl. CPC A01K 67/0278; C12N 9/1241; C12N 9/22; C12N 15/111; C12N 15/90; A01K 2217/05; A01K 2227/10; C12N 2310/20

(57) ABSTRACT

Provided are a nucleic acid construct based on a Cre-LoxP recombination system and a CRISPR gene editing system and use thereof. The Cre-LoxP recombination system comprises a Cre enzyme and a LoxP nucleic acid combination. The LoxP nucleic acid combination comprises TATA-Lox71 and TATA-LoxTC9 sequences, which can only be recombined once under the catalysis of the Cre enzyme. The nucleic acid construct carries an inert “stuffer sequence” with a certain length. The nucleic acid construct can express a plurality of sgRNAs in a low bias manner in vivo, but a same cell can only express one sgRNA, thereby efficiently generating mosaicism whose utilities include accurate and sensitive in-situ CRISPR gene screening and rapid, cost-effective preparation of a single-gene knockout line.

The present application claims the priority of Chinese Patent Application No. 2022105946194 filed on May 27, 2022, the contents of which are incorporated herein by reference in its entirety.

### TECHNICAL FIELD

The present disclosure belongs to the field of gene editing, and specifically relates to the nucleic acid construct based on Cre-LoxP and CRISPR and use thereof in in-situ CRISPR genetic screening and preparation of single-gene perturbation lines.

### BACKGROUND

In vivo genetic screening, CRISPR gene editing: potential and bottleneck. Genetic screening is a crucial strategy for decoding the functions of human genes. Since its inception in 2014, the disruptive genetic screening based on the CRISPR-Cas technology^{[1]} has become a preferred tool for genetic screening. Most CRISPR genetic screens are performed in vitro, typically in tumor cells. However, many physiological and pathological phenomena cannot be (fully) reproduced in vitro, because in vitro systems (including organoids) cannot (fully) mimic the complex cell interactions, immune responses, extracellular matrix structures, and other structural and functional conditions in vivo.

Therefore, in vivo CRISPR screening has been attempted. A conventional approach is to introduce viral sgRNA libraries into tumor cells and then inject them into mice to screen for genes that regulate tumor cell growth, metastasis, or other functions (such as immune tolerance). For example, a genome-scale CRISPR library was introduced into a non-metastatic cancer cell line through viral transduction, and then subcutaneously transplanted into nude mice, resulting in the formation of metastatic foci of some cells. Sequencing results revealed that sgRNAs were enriched at the foci, leading to the discovery of target genes that inhibit tumor metastasis^{[2]}. However, the more important and challenging in vivo screening, which needs to be performed in primary mouse cells, is becoming a frontier in functional genomics. Such in vivo screening falls into two categories, the first being “transplant screening”, where primary cells are first isolated from mice, expanded in vitro, and transduced with viral sgRNA libraries before transplantation in mice. This process is cumbersome, prone to artifacts, and only applicable to a few cell types that are both transplantable and transducible (currently mainly blood cells), thereby having enormous limitations^{[3-7]}. In contrast, in the second category of in vivo screening, termed “in situ screening”, the viral gRNA libraries are directly injected into the mice, which would be ideal because the procedure is simple, the results are reliable, and the method is broadly applicable, thus bypassing the aforementioned limitations. However, the prerequisite for in-situ screening is the efficient delivery of the sgRNA libraries into cells in vivo, which has unfortunately remained an elusive goal. Consequently, in-situ screens have been rarely reported in the literature, and applied so far only to three accessible organs (liver, brain, and lung)^{[8-11]}.

In summary, the severe bottlenecks in the development of CRISPR genetic screening have seriously restricted the deciphering of human gene functions and the diagnosis and treatment of human diseases, therefore a breakthrough is urgently needed.

Cre-Lox recombination system. The Cre-LoxP system is a site-specific recombinant technology, which has been harnessed to create deletion, insertion, translocation, and inversion at target sites on DNA (Mclellan et al., Curr Protoc Mouse Biol, March 2; 7 (1): 1-12). The system consists of Cre-recombinase and its recognition sequence, LoxP (Locus of X-over P1). Cre (Cause recombination) is a tyrosine site-specific recombinase produced by phage P1, which recognizes LoxP, a 34-bp site-specific sequence in the genome of phage P1 consisting of an 8-bp core sequence (spacer) flanked by two 13-bp inverted palindromic repeat symmetric sequences (arms). In genetic manipulation, a pair of LoxP sequences is inserted at both sides of a target sequence; the sequence flanked by a pair of LoxP sequences is called floxed sequence. If the two LoxP sequences flanking the floxed sequence are arranged in the same orientation, the floxed sequence will be deleted upon Cre-mediated recombination, leaving behind a single LoxP site comprising the spacer flanked by the 5′ and 3′ arms derived from the original upstream and downstream LoxP sites, respectively. Two types of LoxP variants have been reported in the literatures, each with its own use. The first type of LoxP variants bear point mutations in the arms, as exemplified by Lox71 and LoxKR3, with point mutations in the 5′ and 3′ arms, respectively, can recombine effectively. But the ensuing hybrid LoxP carries mutations in both arms, thus preventing additional rounds of recombination^{[12]} The second type of LoxP variants bear mutant spacers. For example, replacing the spacer with the TATA box sequence of the U6 promoter can make the LoxP variant (referred to as TATA-Lox) also have TATA box function; inserting TATA-Lox into the U6 promoter does not affect its transcription but makes it have additional recombination function, that is, the modified promoter is bifunctional for transcription and recombination^{[13]}.

iMAP (inducible mosaic animal for perturbation). Over the years, our laboratory has sought to develop iMAP, a new technology aiming at breaking through the above bottlenecks of in-situ screening: using a nucleic acid construct (transgene) based on the Cre-Lox recombination system, multiple sgRNAs are expressed and multiple target genes are knocked out in mice, whereas only one gene is expressed and the other is knocked out in each cell, thus enabling in-situ gene screening without the need for the exogenous sgRNA libraries.

FIG. 1 (including A, B, C, and D) illustrates the principle of iMAP. The core of iMAP is a novel transgene (inserted into the genome by a transposon, and the ITR sequence mediating the insertion is shown in SEQ ID NO: 4), which carries multiple tandemly-linked genetic elements encoding sgRNA (guide RNA encoding sequence, referred to as “guides”), each element floxed with a LoxP variant (as shown in A of FIG. 1). This array of guides is placed downstream of the above U6 promoter with dual functions of transcription and recombination, but only the guide adjacent to the promoter can be expressed; this position is termed Position 0 or P0 and the corresponding guide is termed guide 0 or g0. In contrast, the guides located further downstream (g1, g2, g3 . . . ) are blocked from expression by the transcription termination signal installed immediately following g0. However, in the presence of Cre, the transgene undergoes recombination, allowing all downstream guides the opportunity to move forward to P0 for expression (as shown in B of FIG. 1). Therefore, the mouse can conditionally express all the guides carried in the transgene and knock out their corresponding target genes in the presence of Cas9. However, any given cell can only randomly express one of these guides and knock out one of the target genes (as shown in C of FIG. 1). This allows in-situ CRISPR screening on multiple genes in various cell types throughout the body, thereby overcoming the bottleneck of sgRNA delivery. The iMAP technology has another important application: as the target genes are knocked out among individual sperm in mosaic males, single-gene knockout offspring can be readily sired by the males, thus offering a cost-effective way to generate single-gene knockout lines (as shown in D of FIG. 1).

The premise of iMAP is that only one recombination between the LoxP on the guide and the LoxP on the U6 promoter is allowed. This is because if the downstream LoxP can continue to undergo recombination after the first recombination, then the same cell will successively express a series of guides and knock out the corresponding target genes. However, it is impossible to track which guide has been once expressed, thus causing confusion. Furthermore, repeated recombination can also result in continuous loss of the libraries, eventually deleting the entire libraries and turning it into a “zero-mer” with 0 guide, and generating useless cells lacking any guide, which is wasteful and thus reduces the sensitivity of screening (i.e., a large number of cells are required to perform screening). The solution to the problem is to use a pair of LoxP variants (carrying mutations in the 5′ and 3′ arms, respectively), insert them into U6 promoter and place them at the front end of each guide, and produce a “hybrid” carrying point mutations in both 5′ and 3′ arms after recombination. The key here is that the pair of LoxP variants must be able to recombine effectively, but once one recombination is completed, the subsequent recombination of the resulting hybrid must be terminated.

Serious flaws in the preliminary version of iMAP. The inventors have informally published a preliminary version of iMAP, whose transgene only carries a maximum of 61 guides. More seriously, the following issues greatly limit the application of the preliminary version^{[14]}:

firstly, it is known that for the Lox71-LoxKR3 pair of LoxP variants, Lox71 is able to recombine with LoxKR3, but the ensuing hybrid is inactive^{[12]}. Therefore, the preliminary version utilizes this pair of mutants, while replacing its Spacer with TATA. Unexpectedly, the results of mouse experiments suggest that the TATA-Lox71/KR3 hybrid does not seem to prevent further recombination of the libraries at all, as TAM can induce the production of a large amount of zero-mers and corresponding useless cells (see Chen et al., 2020^{[14]}, such as D of FIG. 2, C of FIG. 3, and C of FIG. 4 in this reference). Subsequent experiments directly demonstrate that the hybrid is indeed capable of recombination, and its efficiency is surprisingly no different from that of the single mutant (as shown in FIG. 2 in this patent application, including A, B, and C). The reason for this anomaly is unknown and may be related to the use of TATA box in the spacer.

Secondly, although each guide in the transgene can be relocated to P0 via recombination, there is a huge bias in the frequencies of recombination, resulting in a severe unevenness in their abundance, with the guides near the 3′ end being the least abundant. This bias in recombination necessitates the use of a large number of target cells to cover the sgRNA library during screening, thus greatly reducing the sensitivity of screening (see Chen et al., 2020^{[14]}, including FIG. 4E in that paper).

In summary, the preliminary version of iMAP technology is seriously flawed in terms of accuracy, sensitivity, and throughput of screening. As a result, the version is of little practical use.

### Content of the Present Invention

In order to improve the accuracy and sensitivity of the preliminary version of iMAP (iMAP v1), the present disclosure provides a nucleic acid construct based on Cre-Lox and CRISPPR-Cas, carrying two novel elements, enabling iMAP to be used for accurate and sensitive in-situ screening and highly efficient and cost-effective preparation of single-gene perturbation lines. Firstly, a new LoxP variant, TATA-LoxTC9, replaces the conventional TATA-LoxKR3, allowing the recombination of TATA-Lox71 undergo only once, rather than multiple times, thus greatly improving the accuracy of screening, while also greatly suppressing the generation of ineffective and useless cells and improving the sensitivity of iMAP; secondly, a certain length of an inert “stuffer” sequence without LoxP is inserted between g0 and g1, which effectively reduces the recombination bias and improves the sensitivity of screening. These innovations also make it possible to prepare the highly efficient and cost-effective single-gene perturbation lines.

The technical solutions of the present disclosure are detailed below:

The first aspect of the present disclosure provides a LoxP nucleic acid pair comprising a TATA-Lox71 sequence and a TATA-LoxTC9 sequence;
wherein the TATA-Lox71 sequence and the TATA-LoxTC9 sequence are shown in SEQ ID NO: 1 and SEQ ID NO: 2, respectively.

The second aspect of the present disclosure provides a Cre-LoxP recombination system comprising a Cre enzyme and the LoxP nucleic acid pair according to the first aspect; the LoxP nucleic acid pair undergoes only one round of Cre-mediated recombination.

The third aspect of the present disclosure provides nucleic acid constructs encoding the Cre-Lox recombination system and a CRISPR gene editing system, which comprises a U6 promoter, sgRNA (guide) expression elements linked in tandem, and an inverted terminal repeat (ITR) sequence of a transposon for inserting the nucleic acid constructs into the genome of a target cell;
wherein the U6 promoter is competent for both transcription and recombination, and carries the TATA-Lox71 sequence; the nucleotide sequences of the TATA-Lox71 sequence and the U6 promoter are shown in SEQ ID NO: 1 and SEQ ID NO: 3, respectively.

The sgRNA expression elements are located downstream of the U6 promoter, and each sgRNA expression element comprises, from the 5′ to the 3′ end, an sgRNA (guide) targeting a target gene, a transcription terminator, and a TATA-LoxTC9 sequence; the nucleotide sequence of the TATA-LoxTC9 sequence is shown in SEQ ID NO: 2.

The LoxP nucleic acid pair according to the first aspect of the present disclosure is capable of one round of Cre-mediated recombination, which induces the expression of sgRNAs, the latter subsequently recruiting Cas proteins or their derivatives to perturb (such as cleave, silence or activate) the target genes.

Using the above nucleic acid constructs, mosaic animals (iMAP) for gene decoding can be generated. In the present disclosure, the gene decoding refers to obtaining functional information about genes through the analysis and interpretation of the entire genome.

In some embodiments of the present disclosure, the numbers of the sgRNA expression elements are more than 2, such as 60 to 150.

In some specific embodiments of the present disclosure, the terminator is T6.

In some embodiments of the present disclosure, both ends of the nucleic acid constructs comprise an inverted terminal repeat (ITR) sequence of a transposon for insertion into the genome of a target cell.

In some specific embodiments of the present disclosure, the transposon is PiggyBac.

In some specific embodiments of the present disclosure, the nucleotide sequence of the inverted terminal repeat is shown in SEQ ID NO: 4.

In some embodiments of the present disclosure, the sgRNA expression elements linked in tandem further comprise a stuffer inserted between the first and second sgRNA expression element, and the stuffer is an inert random sequence incapable of recombination.

In some embodiments of the present disclosure, the length of the stuffer is 0.5 kb to 10 kb, such as 2 kb.

The fourth aspect of the present disclosure provides a recombinant expression vector, which comprises the LoxP nucleic acid pair according to the first aspect, the Cre-LoxP recombination system according to the second aspect, or the nucleic acid construct according to the third aspect.

In some embodiments of the present disclosure, the recombinant expression vector further comprises a nucleotide sequence encoding a Cre enzyme and/or a Cas protein or derivatives thereof.

In the present disclosure, both the Cre enzyme and Cas protein can be conventional in the art; the Cre enzyme is, for example, a wild-type Cre enzyme or CreER, and the Cas protein is, for example, a Cas9 protein.

The fifth aspect of the present disclosure provides a recombinant cell, which comprises the LoxP nucleic acid pair according to the first aspect, the Cre-LoxP recombination system according to the second aspect, the nucleic acid construct according to the third aspect, or the recombinant expression vector according to the fourth aspect.

In some embodiments of the present disclosure, the cell is derived from a mammalian cell line.

In some preferred embodiments of the present disclosure, the cell is derived from mice, rats, or rabbits.

The sixth aspect of the present disclosure provides a method for preparing single-gene perturbation animal lines, which comprises:

using the nucleic acid construct according to the third aspect, recombining TATA-Lox71 at the U6 promoter with TATA-LoxTC9 on the sgRNA expression elements using Cre enzyme, so that the sgRNA is randomly expressed in germ cells of an animal in vivo, and then deriving an offspring line expressing the same sgRNA throughout the body via natural reproduction, followed by introducing a transgene expressing a Cas protein or derivatives thereof into the offspring line to obtain single-gene perturbation lines; or, generating a mosaic animal with random gene perturbation, and then breeding single-gene perturbation lines.

The seventh aspect of the present disclosure provides a use of the LoxP nucleic acid pair according to the first aspect, the Cre-LoxP recombination system according to the second aspect, the nucleic acid construct according to the third aspect, the recombinant expression vector according to the fourth aspect, or the cell according to the fifth aspect in in-situ CRISPR genetic screening or preparation of single-gene perturbation lines.

On the basis of common knowledge in the art, the above preferred conditions can be arbitrarily combined to obtain preferred examples of the present disclosure.

The reagents and raw materials used in the present disclosure are all commercially available.

The positive and progressive effects of the present disclosure are as follows:

The present disclosure uses TATA-LoxTC9 and stuffer to overcome the two key flaws of the preliminary version and effectively improve the accuracy and sensitivity of iMAP, making iMAP a practical tool for decoding gene functions, whose applications include in-situ genetic screening and preparation of single-gene perturbation lines.

In addition, the upgraded version of the nucleic acid constructs of the present disclosure can carry 100 sgRNAs (contrary to only 60 in the preliminary version), which has been stably inherited for 13 generations (contrary to only 5 in the preliminary version).

### BRIEF DESCRIPTION OF THE DRAWINGS

FIG. 1 illustrates the iMAP principle.
(A) Transgene before recombination. Arrows indicate productive recombination capable of inducing guide expression. Recombination among TATA-LoxTC9 is also possible, but unable to induce guide expression (not shown).
(B) Transgene after recombination. Ubc-CreER is a transgene broadly expressing CreER, wherein CreER is a fusion protein of Cre and ER that is activated by Tamoxifen (TAM). PCR primers a/b target U6 and the scaffold of the guide, respectively, enabling amplification of all guides located at P0.
(C) Mosaic mice. CAG-Cas9 is a transgene broadly expressing Cas9. The dots indicate the cells whose target genes have been knocked out.
(D) iMAP allows efficient and cost-effective preparation of single-gene knockout lines.

FIG. 2 depicts the development of LoxTC9.
(A) LoxP sequence. The wild-type Spacer is replaced by TATA (GTATAAAT) with the omission of “TATA” in the nomenclature of LoxP.
(B) Potential recombination scenario of 61-guide transgene^{[14]}. a/b, PCR primer pair used to amplify the transgene, wherein primer a was also used for Sanger sequencing of the PCR products to reveal changes in the Lox71 sequence.
(C) Results of the 61-guide mice experiment.
(D) In vitro assay quantifying various LoxP variants.
(E) In vivo confirming of the stability of the Lox71/TC9 hybrid (SEQ ID NO: 28).

FIG. 3 depicts the stuffer-based strategy.
(A) Schematic illustration of conventional (left) and novel (right) transgene configurations, the latter bearing a 2-kb stuffer inserted between g0 and g1.
(B) Abundance of various guides at P0 after recombination. Guides are arranged based on their positions on the array in the resting state.

FIG. 4 diagrams the workflow for assembling the 91-guide transgene.

### DETAILED DESCRIPTION OF THE PREFERRED EMBODIMENT

The present disclosure is further illustrated below by means of examples, but it is not thereby limited to the scope of the described embodiments. The experimental methods for which the specific conditions are not specified in the following embodiments, shall be carried out according to the conventional methods and conditions, or according to the manufacturers' instructions.

Table 1 below lists the consumables used in the experiment, including molecular reagents, organic reagents, enzymes, kits, and antibodies.

TABLE 1
Experimental consumables

| Name | Corporation/Cat. No. |
|---|---|
| Tamoxifen | TargetMol/T6906 |
| Kanamycin sulfate | Solarbio/K8020 |
| FastPure Plasmid Mini Kit | Vazyme/DC201-01 |
| KOD One ™ PCR Master Mix -Blue- | TOYOBO/KMM-201 |
| Mouse Direct PCR Kit (For Genotyping) | Bimake/B40013 |
| NEBNext Ultra II Q5 Master Mix | NEB/M0544S |
| NEBuilder HiFi DNA Assembly Master Mix | NEB/E2621L |
| Q5 High-Fidelity PCR Kit | NEB/E0555L |

The sources of experimental cells and animals are as follows:

### 1. Bacterial Strains

NEB® Stable Competent E. coli (NEB, C3040I) is an E. coli strain used for plasmid cloning.

### 2. Cells

HEK293T cells (ATCC: CRL-11268) are a human renal epithelial cell line.

Mouse N2a cell line (ATCC: CCL-131).

### 3. Mice

3.1 CAG-Cas9 (JAX: 028555), transgenic mice that broadly express Cas9, purchased from Jackson Laboratory and housed at the Southern Model Animal Center.
3.2 UBC-Cre-ERT2 (JAX: 007179), transgenic mice that broadly express Cre-ERT2, purchased from Jackson Laboratory and housed at the Southern Model Animal Center.
3.3 100-guide, iMAP transgenic mice without the stuffer. The transgene was constructed in house and the transgenic mice produced at the Southern Model Animal Center.
3.4 91-guide, iMAP transgenic mice carrying the stuffer. The transgene was constructed in house and the transgenic mice produced at the Southern Model Animal Center. (see Example 3 for details).

### Example 1: Development of TATA-LoxTC9

The preliminary version of the iMAP transgene consists of 61 guides linked in tandem (“61-guide”), wherein TATA-Lox71 (SEQ ID NO: 1) is embedded in the U6 promoter (SEQ ID NO: 3) and placed at the end of the transgene, while TATA-LoxKR3 (as shown in A of FIG. 2) is inserted into the transgene (as shown in the top of B of FIG. 2). The inventors previously discovered that in mice, the transgene is prone to excessive recombination, resulting in the loss of the entire library and the production of a large number of useless cells (Chen et al., 2020). The inventors speculated that, the hybrid resulting from Lox71-KR3 recombination is not actually non-functional as predicted from the literature, but could rather undergo repeated rounds of recombination with downstream LoxKR3, thus approaching and recombining with Lox71 at the end of the transgene by continuously deleting guides, which culminates in the complete deletion of the entire array and the subsequent reversion of the hybrid to Lox71 (as shown in the bottom of B of FIG. 2). Therefore, the conversion of the hybrid to Lox71 is a result as well as a reflection and evidence of the above recombination process. To test this hypothesis, the following experiment was conducted in the present disclosure. A 61-guide transgenic mouse carrying the Cre-ER was treated with TAM, and the tail DNA was collected at different time points before analysis by PCR-Sanger sequencing. As shown in C of FIG. 2, within 2 days after TAM treatment, approximately 50% of the Lox71 had been recombined to form the Lox71/KR3 hybrid; importantly, the latter was then subsequently converted back to Lox71. This result is inconsistent with literature reports. The reason for the discrepancy is unknown, but maybe related to the replacement of the LoxP wild-type spacer with the TATA box.

The pair of TATA-LoxP variants used in iMAP must meet two criteria: efficient recombination between the two but inability of the resultant hybrid LoxP to undergo further recombination. Many LoxP variants were designed and screened in vitro, followed by in vivo validation of the top candidates, which led finally to the identification of a pair of variants (TATA-LoxTC9 and TATA-Lox71) that satisfy both criteria (as shown in A of FIG. 2). The experiment is detailed below:
(1) In vitro characterization of LoxP variants (as shown in D of FIG. 2). A LoxP reporter carrying mCherry and GFP was designed in the present disclosure, wherein mCherry is continuously expressed to serve as an internal reference, while GFP is only induced after successful recombination of the LoxP pair concomitant with the deletion of the intervening transcription terminating signal (STOP), thus reflecting the recombination efficiency of LoxP. The reporter and CreER-expression plasmids were co-transfected into the mouse N2a cell line, and the fluorescence detected by flow cytometry 2 days later. In the control group carrying wild-type LoxP, 71% of the transfected cells expressed GFP (as shown in Plot 2 of D of FIG. 2). The Lox71-LoxKR3 recombination was slightly less efficient (52% GFP, Plot 3). Surprisingly, the Lox71/KR3 hybrid-LoxKR3 recombination was just as efficient (56% GFP, Plot 4) compared to that of the single variant, explaining the 61-guide mouse phenotype as shown in B of FIG. 2. In sharp contrast, the novel TATA-LoxTC9 developed in the present disclosure was only moderately less efficient than wild-type TATA-Lox when recombining with Lox71 (40% vs. 71% GFP, Plot 5), but the ensuing hybrid (Lox71/TC9) was essentially inactive (only 8% GFP, Plot 6), suggesting that the Lox71-LoxTC9 pair may be suitable for iMAP.
(2) In vivo validation of the Lox71-LoxTC9 combination (as shown in E of FIG. 2). In the present disclosure, 100-guide transgenic mice carrying LoxTC9 as shown in A of FIG. 3 were first generated. Subsequently, the offspring expressing a single guide was derived, and the specific steps are as follows:
(2.1) A male double-transgenic for the above-mentioned 100-guide and also the (bc-CreER transgenes was exposed to TAM via oral gavage (0.1 mg/g once daily for a total of 3 days, and then 0.2 mg/g once daily for a total of 3 days), so that numerous recombinant transgenes were generated in mouse, but each sperm could only carry one of them at random. In addition, all transgenic 3′ ends were tagged with g99-Cd45 (targeting Cd45), which lacks 3′ LoxP and thus undeletable, facilitating the analysis of recombination (more details see further).
(2.2) The male was then mated with Ubc-CreER transgenic females to sire double transgenic offspring lines carrying both a single recombinant iMAP transgene and the (bc-CreER transgene. The double transgenic line analyzed in the present disclosure carried g36-Ets2 (targeting Ets2).
(2.3) The g36-Ets2; (bc-CreER mouse was repeatedly challenged with TAM (0.2 mg/g once daily for 6 consecutive days, and repeated after an interval of 3 days) before the tail DNA was analyzed by PCR-Sanger sequencing (primers in B of FIG. 2). If and only if the Lox71/TC9 hybrid is unstable, g99 will replace g35 in at least some of the cells (as shown in E of FIG. 2, left). Remarkably, g99-Cd45 signal was undetectable despite repeated stimulation with TAM, indicating the stability of the hybrid, thus validating the conclusion of the in vitro experiment (as shown in E of FIG. 2, right).

### Example 2: Development of the Stuffer-Based Strategy

As shown in A of FIG. 3, the abundance of each guide that had been moved forward to P0 after 100-guide recombination was first detected in the present disclosure. The specific steps are: after gastric gavage of TAM, the tail DNA was collected, P0 guide was amplified via PCR, and high-throughput sequencing was performed. Before recombination, P0 only contained g0 (i.e., the abundance of g0 was 100%; data not shown). After recombination, g0 dropped to ˜ 10%, while g1 to g99 all emerged at P0 but at uneven frequencies, g2 to g10 exhibited the highest abundances (g2 was as high as 10%). The downstream guides showed a gradually decreasing in general in their abundances, with the least abundant comprising only 0.14% of the total P0 guides (a 71-fold difference from g2), but the tail of the transgene tended to tilt up again, making the entire curve more or less U-shaped, which was similar to the previously published 61-guide (carrying LoxKR3) (as shown in B of FIG. 3, 100-guide). The inventors speculated that g2 to g10 were over-represented perhaps because these guides are closer to Lox71, therefore they preferentially recombined with Lox71. Considering that g10 is 1.8 kb away from Lox71, an inert sequence with a similar length but lacking LoxP (“stuffer”), if inserted after g0, might force Lox71 to skip the hotspot to recombine with the downstream LoxTC9, thereby mitigating the recombination bias. To test this hypothesis, the 91-guide transgene was assembled in the present disclosure; the workflow for transgene assembly is detailed in Example 3. The results showed that the stuffer indeed markedly improved the evenness in the sgRNA representation, in that g2 to g18 have become less abundant, while the abundances of its downstream guides increased after the insertion compared with that before the insertion, leading to an increase of the least abundant guide from 0.14% to 0.42% of the total, which amounted to 3-fold (300%) improvement in sensitivity. Of note, in the 100-guide line, g1 was paradoxically less abundant than g2, perhaps because the adjacent U6 promoter interfered with its recombination; the stuffer eliminated this anomaly and further optimized iMAP.

It is also noteworthy that like the 100-guide, 91-guide similarly exhibited a “tail-up” pattern for the following possible reasons. Presumably, guides in the transgene were not only relocated to P0 through LoxTC9-Lox71 recombination, but also deleted through recombination between LoxTC9 sites, the two competing processes exerting opposite effects on the abundance of guides at P0. The efficiency of deletion of a given guide depends on the number of LoxTC9 sites on either side. The lack of LoxTC9 (or other LoxP sites) at the 3′ end of 100-guide and 91-guide prevented the terminal guide from deletion while hampering the deletion of its adjacent guides, hence their increased abundances at P0.

### Example 3: Construction of the 91-Guide Line

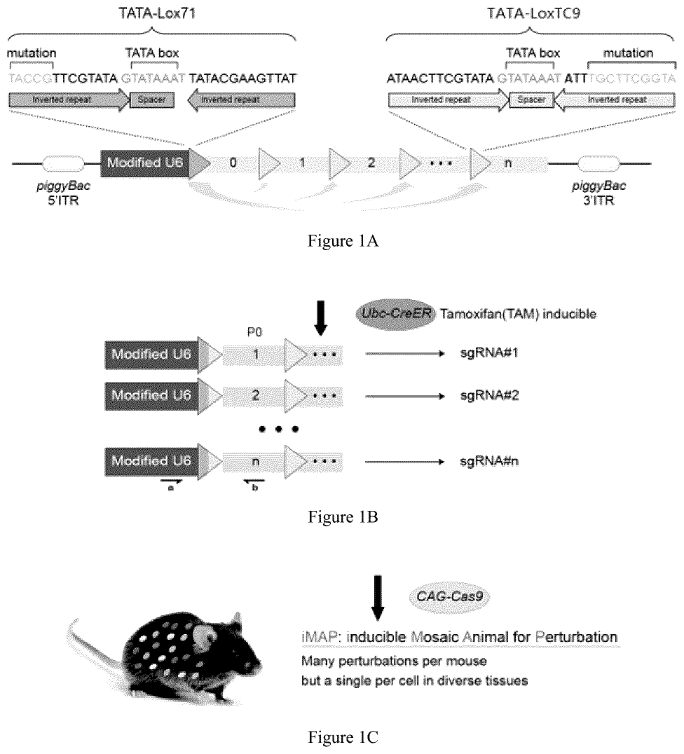

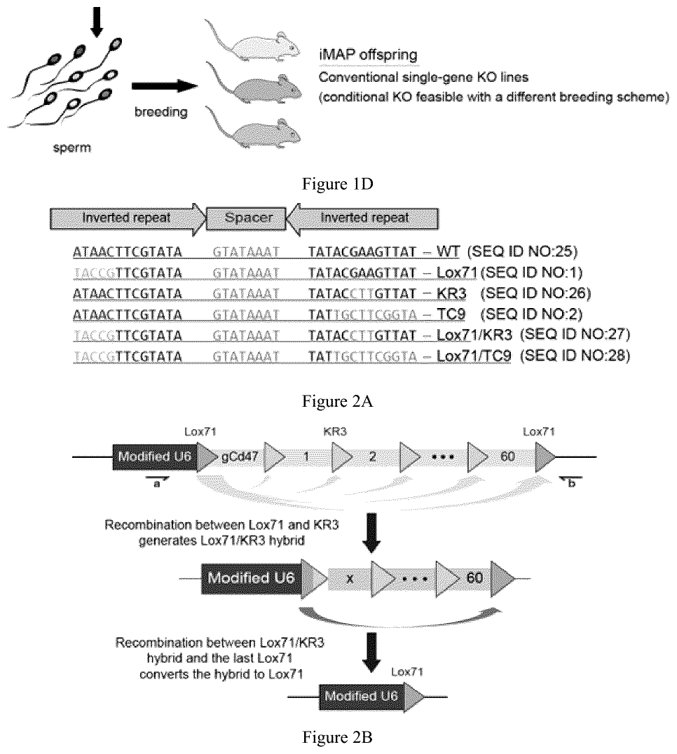

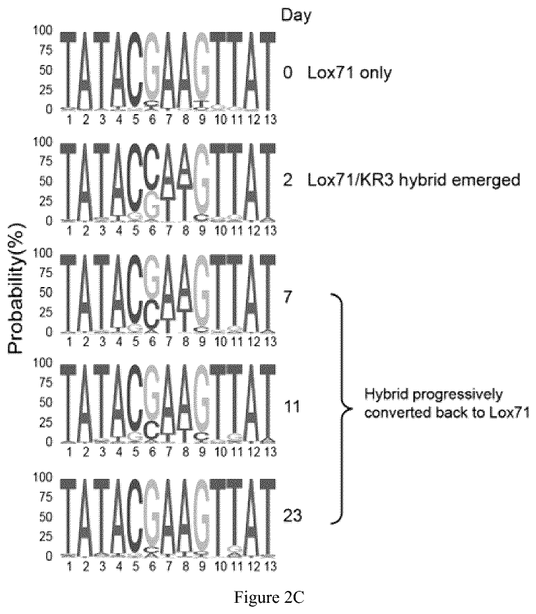

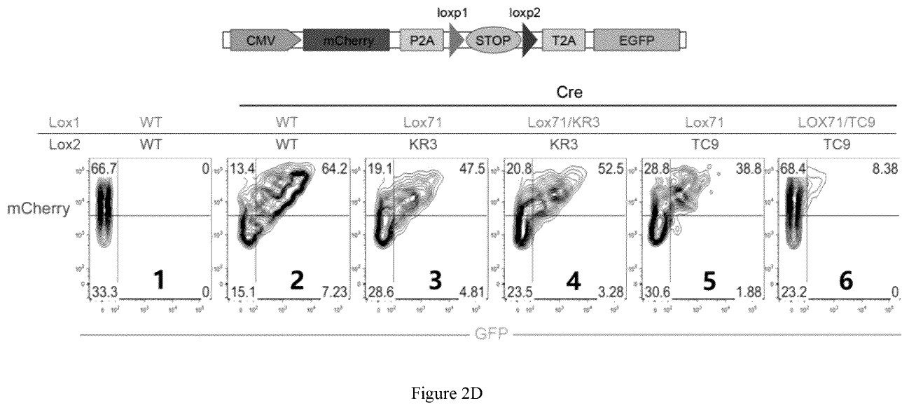

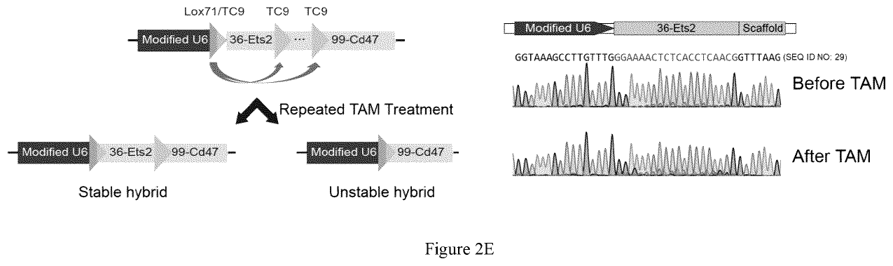

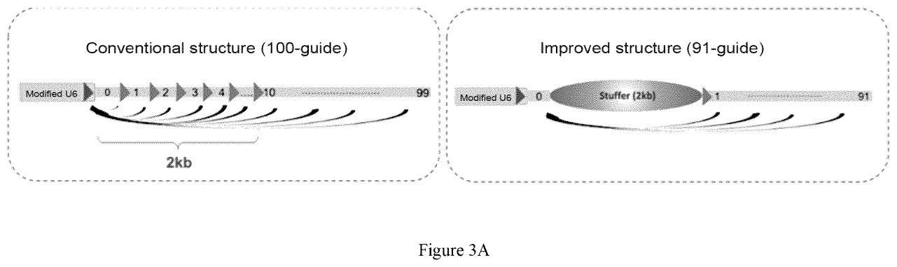

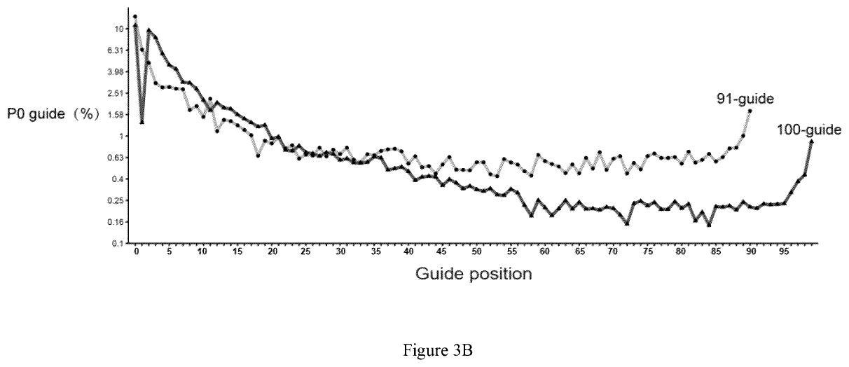

The transgene carries 91 guides targeting various genes involved in RNA modification except for g0 and for 8 negative control guides. In addition, a 2-kb stuffer was inserted between g0 and g1 to reduce the recombination bias. The workflow for assembling the guide array is depicted in FIG. 4 and detailed below.
(1) Generation of 90 sgRNA-expression fragments by PCR. The PCR templates comprising a Cas9 sgRNA direct repeat (Scaffold) and a transcription termination signal (Stop) upstream of TATA-LoxTC9 (SEQ ID NO: 2). Using 90 pairs of primers, 90 fragments were amplified, each flanked by a BsaI recognition site coupled to a four-base-pair cleavage site, the latter serving as a linker for directional ligation. The 90 fragments were divided into 9 groups with 10 in each group, and equal amounts of the fragments were mixed and then purified. PCR amplification conditions: NEB Q5 2× mix system (20 μL), 98° C. for 3 minutes, (98° C. for 5 seconds, 65° C. for 5 seconds, 72° C. for 20 seconds)×30 cycles, 72° C. for 2 minutes.
(2) Preparation of intermediate vectors: 9 fragments were PCR-amplified using KOD from the pUC57-Amp (SEQ ID NO: 5) template, and subsequently purified. The PCR primers are shown in Table 2.
(3) The 9 groups of PCR products described above were each mixed with the corresponding intermediate vectors, and subjected to Golden Gate cloning (NEB), which ligated the 10 fragments through the BsaI-generated linkers and inserted the ligation products into the corresponding intermediate vectors. The Golden Gate reaction condition is: (37° C. for 5 minutes→16° C. for 5 minutes)×30 cycles followed by 60° C. for 5 minutes.
(4) 1 μL of the Golden Gate reaction product was mixed with 10 μL of competent cells (NEB Stbl II), which were incubated on ice for 30 minutes, heat shocked for 30 seconds, chilled on ice for 2 minutes before addition of 90 μL of LB medium and shaking at 30° C. for 30 minutes. 100 μL of bacterial solution was then spread on ampicillin-containing agar plates. After incubation overnight at 30° C., clones were picked for sequencing to obtain 9 correct 10-guide plasmids (SEQ ID NO: 5).
(5) The nine 10-guide plasmids were mixed with the destination plasmid. Golden Gate cloning was used to release and ligate the nine 10-guide fragments before insertion into the destination plasmid through the Esp3I restriction sites (located downstream of the stuffer), thus generating the 91-guide plasmid, which was sequence-verified. The destination plasmid carries the key element “U6-g0-stuffer”, and the entire plasmid sequence is shown in SEQ ID NO: 6.
(6) The 91-guide plasmid above was mixed with PBase mRNA and injected into fertilized eggs to obtain 91-guide mice at Shanghai Model Organisms Center.

### REFERENCES

1. Shalem, O., Sanjana, N. E., Hartenian, E., Shi, X., Scott, D. A., Mikkelsen, T. S., Heckl, D., Ebert, B. L., Root, D. E., Doench, J. G., et al. (2014). Genome-Scale CRISPR-Cas9 Knockout Screening in Human Cells. Science 343, 84-87. 10.1126/science.1247005.
2. Chen, S., Sanjana, N. E., Zheng, K., Shalem, O., Lee, K., Shi, X., Scott, D. A., Song, J., Pan, J. Q., Weissleder, R., et al. (2015). Genome-wide CRISPR Screen in a Mouse Model of Tumor Growth and Metastasis. Cell 160, 1246-1260. 10.1016/j.cell.2015.02.038.
3. Chen, Z., Arai, E., Khan, O., Zhang, Z., Ngiow, S. F., He, Y., Huang, H., Manne, S., Cao, Z., Baxter, A. E., et al. (2021). In vivo CD8+ T cell CRISPR screening reveals control by Flil in infection and cancer. Cell 184, 1262-1280.e22. 10.1016/j.cell.2021.02.019.
4. Dong, M. B., Wang, G., Chow, R. D., Ye, L., Zhu, L., Dai, X., Park, J. J., Kim, H. R., Errami, Y., Guzman, C. D., et al. (2019). Systematic Immunotherapy Target Discovery Using Genome-Scale In Vivo CRISPR Screens in CD8 T Cells. Cell 178, 1189-1204.e23. 10.1016/j.cell.2019.07.044.
5. Huang, H., Zhou, P., Wei, J., Long, L., Shi, H., Dhungana, Y., Chapman, N. M., Fu, G., Saravia, J., Raynor, J. L., et al. (2021). In vivo CRISPR screening reveals nutrient signaling processes underpinning CD8+ T cell fate decisions. Cell 184, 1245-1261.e21. 10.1016/j.cell.2021.02.021.
6. LaFleur, M. W., Nguyen, T. H., Coxe, M. A., Yates, K. B., Trombley, J. D., Weiss, S. A., Brown, F. D., Gillis, J. E., Coxe, D. J., Doench, J. G., et al. (2019). A CRISPR-Cas9 delivery system for in vivo screening of genes in the immune system. Nat. Commun. 10, 1-10. 10.1038/s41467-019-09656-2.
7. Wei, J., Long, L., Zheng, W., Dhungana, Y., Lim, S. A., Guy, C., Wang, Y., Wang, Y.-D., Qian, C., Xu, B., et al. (2019). Targeting REGNASE-1 programs long-lived effector T cells for cancer therapy. Nature 576, 471-476. 10.1038/s41586-019-1821-z.
8. Jin, X., Simmons, S. K., Guo, A., Shetty, A. S., Ko, M., Nguyen, L., Jokhi, V., Robinson, E., Oyler, P., Curry, N., et al. (2020). In vivo Perturb-Seq reveals neuronal and glial abnormalities associated with autism risk genes. Science 370. 10.1126/science.aaz6063.
9. Rogers, Z. N., McFarland, C. D., Winters, I. P., Seoane, J. A., Brady, J. J., Yoon, S., Curtis, C., Petrov, D. A., and Winslow, M. M. (2018). Mapping the in vivo fitness landscape of lung adenocarcinoma tumor suppression in mice. Nat. Genet. 50, 483-486. 10.1038/s41588-018-0083-2.
10. Wang, G., Chow, R. D., Ye, L., Guzman, C. D., Dai, X., Dong, M. B., Zhang, F., Sharp, P. A., Platt, R. J., and Chen, S. (2018). Mapping a functional cancer genome atlas of tumor suppressors in mouse liver using AAV-CRISPR-mediated direct in vivo screening. Sci. Adv. 4, eaao5508. 10.1126/sciadv.aao5508.
11. Wertz, M. H., Mitchem, M. R., Pineda, S. S., Hachigian, L. J., Lee, H., Lau, V., Powers, A., Kulicke, R., Madan, G. K., Colic, M., et al. (2020). Genome-wide In Vivo CNS Screening Identifies Genes that Modify CNS Neuronal Survival and mHTT Toxicity. Neuron 106, 76-89.e8. 10.1016/j.neuron.2020.01.004.
12. Araki, K., Okada, Y., Araki, M., and Yamamura, K. (2010). Comparative analysis of right element mutant lox sites on recombination efficiency in embryonic stem cells. BMC Biotechnol. 10, 29. 10.1186/1472-6750-10-29.
13. Ventura, A., Meissner, A., Dillon, C. P., McManus, M., Sharp, P. A., Van Parijs, L., Jaenisch, R., and Jacks, T. (2004). Cre-lox-regulated conditional RNA interference from transgenes. Proc. Natl. Acad. Sci. 101, 10380-10385. 10.1073/pnas.0403954101.
14. Chen, Y., Sun, J., Mao, S., Jing, Z., Zang, Y., Xia, J., Sun, J., and Chi, T. (2020). Novel mosaic mice with diverse applications. bioRxiv, 2020.03.21.001388. 10.1101/2020.03.21.001388.

The sequences used in the present disclosure are as follows:

TATA-Lox71 (SEQ ID NO: 1):
TACCGTTCGTATAGTATAAATTATACGAAGTTAT
TATA-LoxTC9 (SEQ ID NO: 2):
ATAACTTCGTATAGTATAAATTATTGCTTCGGTA
U6 promoter (SEQ ID NO: 3):
CGACGCCGCCATCTCTAGGCCCGCGCCGGCCCCCTCGCACAGACTTGTG
GGAGAAGCTCGGCTACTCCCCTGCCCCGGTTAATTTGCATATAATATTT
CCTAGTAACTATAGAGGCTTAATGTGCGATAAAAGACAGATAATCTGTT
CTTTTTAATACTAGCTACATTTTACATGATAGGCTTGGATTTCTATAAG
AGATACAAATACTAAATTATTATTTTAAAAAACAGCACAAAAGGAAACT
CACCCTAACTGTAAAGTAATTTACCGTTCGTATAGTATAAATTATACGA
AGTTATAAGCCTTGTTTG
ITR (SEQ ID NO: 4):
ATTCTTGAAATATTGCTCTCTCTTTCTAAATAGCGCGAATCCGTCGCTG
TGCATTTAGGACATCTCAGTCGCCGCTTGGAGCTCCCGTGAGGCGTGCT
TGTCAATGCGGTAAGTGTCACTGATTTTGAACTATAACGACCGCGTGAG
TCAAAATGACGCATGATTATCTTTTACGTGACTTTTAAGATTTAACTCA
TACGATAATTATATTGTTATTTCATGTTCTACTTACGTGATAACTTATT
ATATATATATTTTCTTGTTATAGATAGCCGATAAAAGTTTTGTTACTTT
ATAGAAGAAATTTTGAGTTTTTGtTTTTTTTTAATAAATAAATAAACAT
AAATAAATTGTTTGTTGAATTTATTATTAGTATGTAAGTGTAAATATAA
TAAAACTTAATATCTATTCAAATTAATAAATAAACCTCGATATACAGAC
CGATAAAACACATGCGTCAATTTTACgCATGATTATCTTTAACGTACGT
CACAATATGATTATCTTTCTAGGGTTAA
10-guide transition vector (SEQ ID NO: 5):
AGCAACGCGGCCTTTTTACGGTTCCTGGCCTTTTGCTGGCCTTTTGCTC
ACATGTTCTTTCCTGCGTTATCCCCTGATTCTGTGGATAACCGTGGTCT
CAGAAGGATATCCGGTTGAGACCCACCGTCATCACCGAAACGCGCGATG
CAGCTCTGGCCCGTGTCTCAAAATCTCTGATGTTACATTGCACAAGATA
AAAATATATCATCATGAACAATAAAACTGTCTGCTTACATAAACAGTAA
TACAAGGGGTGTTATGAGCCATATTCAACGGGAAACGTCGAGGCCGCGA
TTAAATTCCAACATGGATGCTGATTTATATGGGTATAAATGGGCTCGCG
ATAATGTCGGGCAATCAGGTGCGACAATCTATCGCTTGTATGGGAAGCC
CGATGCGCCAGAGTTGTTTCTGAAACATGGCAAAGGTAGCGTTGCCAAT
GATGTTACAGATGAGATGGTCAGACTAAACTGGCTGACGGAATTTATGC
CTCTTCCGACCATCAAGCATTTTATCCGTACTCCTGATGATGCATGGTT
ACTCACCACTGCGATCCCCGGAAAAACAGCATTCCAGGTATTAGAAGAA
TATCCTGATTCAGGTGAAAATATTGTTGATGCGCTGGCAGTGTTCCTGC
GCCGGTTGCATTCGATTCCTGTTTGTAATTGTCCTTTTAACAGCGATCG
CGTATTTCGTCTGGCTCAGGCGCAATCACGAATGAATAACGGTTTGGTT
GATGCGAGTGATTTTGATGACGAGCGTAATGGCTGGCCTGTTGAACAAG
TCTGGAAAGAAATGCATAAACTTTTGCCATTCTCACCGGATTCAGTCGT
CACTCATGGTGATTTCTCACTTGATAACCTTATTTTTGACGAGGGGAAA
TTAATAGGTTGTATTGATGTTGGACGAGTCGGAATCGCAGACCGATACC
AGGATCTTGCCATCCTATGGAACTGCCTCGGTGAGTTTTCTCCTTCATT
ACAGAAACGGCTTTTTCAAAAATATGGTATTGATAATCCTGATATGAAT
AAATTGCAGTTTCATTTGATGCTCGATGAGTTTTTCTAATCAGAATTGG
TTAATTGGTTGTAACATTATTCAGATTGGGCTTGATTTAAAACTTCATT
TTTAATTTAAAAGGATCTAGGTGAAGATCCTTTTTGATAATCTCATGAC
CAAAATCCCTTAACGTGAGTTTTCGTTCCACTGAGCGTCAGACCCCGTA
GAAAAGATCAAAGGATCTTCTTGAGATCCTTTTTTTCTGCGCGTAATCT
GCTGCTTGCAAACAAAAAAACCACCGCTACCAGCGGTGGTTTGTTTGCC
GGATCAAGAGCTACCAACTCTTTTTCCGAAGGTAACTGGCTTCAGCAGA
GCGCAGATACCAAATACTGTTCTTCTAGTGTAGCCGTAGTTAGGCCACC
ACTTCAAGAACTCTGTAGCACCGCCTACATACCTCGCTCTGCTAATCCT
GTTACCAGTGGCTGCTGCCAGTGGCGATAAGTCGTGTCTTACCGGGTTG
GACTCAAGACGATAGTTACCGGATAAGGCGCAGCGGTCGGGCTGAACGG
GGGGTTCGTGCACACAGCCCAGCTTGGAGCGAACGACCTACACCGAACT
GAGATACCTACAGCGTGAGCTATGAGAAAGCGCCACGCTTCCCGAAGGG
AGAAAGGCGGACAGGTATCCGGTAAGCGGCAGGGTCGGAACAGGAGAGC
GCACGAGGGAGCTTCCAGGGGGAAACGCCTGGTATCTTTATAGTCCTGT
CGGGTTTCGCCACCTCTGACTTGAGCGTCGATTTTTGTGATGCTCGTCA
GGGGGGCGGAGCCTATGGAAAAACGCC
91-guide target plasmid (SEQ ID NO: 6):
AAATCAATCTAAAGTATATATGAGTAAACTTGGTCTGACAGTTACCAAT
GCTTAATCAGTGAGGCACCTATCTCAGCGATCTGTCTATTTCGTTCATC
CATAGTTGCCTGACTCCCCGTCGTGTAGATAACTACGATACGGGAGGGC
TTACCATCTGGCCCCAGTGCTGCAATGATACCGCGAGATCCACGCTCAC
CGGCTCCAGATTTATCAGCAATAAACCAGCCAGCCGGAAGGGCCGAGCG
CAGAAGTGGTCCTGCAACTTTATCCGCCTCCATCCAGTCTATTAATTGT
TGCCGGGAAGCTAGAGTAAGTAGTTCGCCAGTTAATAGTTTGCGCAACG
TTGTTGCCATTGCTACAGGCATCGTGGTGTCACGCTCGTCGTTTGGTAT
GGCTTCATTCAGCTCCGGTTCCCAACGATCAAGGCGAGTTACATGATCC
CCCATGTTGTGCAAAAAAGCGGTTAGCTCCTTCGGTCCTCCGATCGTTG
TCAGAAGTAAGTTGGCCGCAGTGTTATCACTCATGGTTATGGCAGCACT
GCATAATTCTCTTACTGTCATGCCATCCGTAAGATGCTTTTCTGTGACT
GGTGAGTACTCAACCAAGTCATTCTGAGAATAGTGTATGCGGCGACCGA
GTTGCTCTTGCCCGGCGTCAATACGGGATAATACCGCGCCACATAGCAG
AACTTTAAAAGTGCTCATCATTGGAAAACGTTCTTCGGGGCGAAAACTC
TCAAGGATCTTACCGCTGTTGAGATCCAGTTCGATGTAACCCACTCGTG
CACCCAACTGATCTTCAGCATCTTTTACTTTCACCAGCGTTTCTGGGTG
AGCAAAAACAGGAAGGCAAAATGCCGCAAAAAAGGGAATAAGGGCGACA
CGGAAATGTTGAATACTCATACTCTTCCTTTTTCAATATTATTGAAGCA
TTTATCAGGGTTATTGTCTCATGAGCGGATACATATTTGAATGTATTTA
GAAAAATAAACAAATAGGGGTTCCGCGCACATTTCCCCGAAAAGTGCCA
CCTGACGTCTAAGAAACCATTATTATCATGACATTAACCTATAAAAATA
GGCGTATCACGAGGCCCTTTAGGCCTTTAACCCTAGAAAGATAGTCTGC
GTAAAATTGACGCATGCATTCTTGAAATATTGCTCTCTCTTTCTAAATA
GCGCGAATCCGTCGCTGTGCATTTAGGACATCTCAGTCGCCGCTTGGAG
CTCCCGTGAGGCGTGCTTGTCAATGCGGTAAGTGTCACTGATTTTGAAC
TATAACGACCGCGTGAGTCAAAATGACGCATGATTATCTTTTACGTGAC
TTTTAAGATTTAACTCATACGATAATTATATTGTTATTTCATGTTCTAC
TTACGTGATAACTTATTATATATATATTTTCTTGTTATAGATAGCTTCG
ATACCGTCGGCTCGAGAATGCATCTAGAGGATCCCCACAGGTCCGACGC
CGCCATCTCTAGGCCCGCGCCGGCCCCCTCGCACAGACTTGTGGGAGAA
GCTCGGCTACTCCCCTGCCCCGGTTAATTTGCATATAATATTTCCTAGT
AACTATAGAGGCTTAATGTGCGATAAAAGACAGATAATCTGTTCTTTTT
AATACTAGCTACATTTTACATGATAGGCTTGGATTTCTATAAGAGATAC
AAATACTAAATTATTATTTTAAAAAACAGCACAAAAGGAAACTCACCCT
AACTGTAAAGTAATTTACCGTTCGTATAGTATAAATTATACGAAGTTAT
AAGCCTTGTTTGAATGTCTCAGACCATATGGGGTTTAAGAGCTATGCTG
GAAACAGCATAGCAAGTTTAAATAAGGCTAGTCCGTTATCAACTTGAAA
AAGTGGCACCGAGTCGGTGCTTTTTTTGGGAAGTTCCTATTCCGAAGTT
CCTATTCTtcAAATAGTATAGGAACTTCGAACGCTGACGTCATCAACCC
GCTCCAAGGAATCGCGGGCCCAGTGTCACTAGGCGGGAACACCCAGCGC
GCGTGCGCCCTGGCAGGAAGATGGCTGTGAGGGACAGGGGAGTGGCGCC
CTGCAATATTTGCATGTCGCTATGTGTTCTGGGAAATCACCATAAACGT
GAAATGTCTTTGGATTTGGGAATCTTcgAAGTTCTGTATGAGACCACGA
AACACCGGAATTCGCCACCATGGTGAGCAAGGGCGAGGCCGTGATCAAG
GAGTTCATGAGGTTTAAGGTGCACATGGAGGGCAGCATGAACGGCCACG
AGTTCGAGATCGAGGGAGAGGGAGAGGGCAGACCCTACGAGGGCACCCA
GACAGCTAAGCTGAAGGTGACCAAGGGCGGACCACTGCCCTTTAGCTGG
GACATCCTGTCCCCTCAGTTCATGTACGGCAGCAGGGCCTTCATCAAGC
ACCCTGCTGACATCCCAGATTACTACAAGCAGTCTTTCCCAGAGGGCTT
TAAGTGGGAGAGAGTGATGAACTTCGAGGACGGCGGAGCCGTGACCGTG
ACACAGGACACCTCTCTGGAGGATGGAACACTGATCTACAAGGTGAAGC
TGCGGGGAACAAACTTTCCCCCTGATGGCCCAGTGATGCAGAAGAAAAC
CATGGGATGGGAGGCCAGCACAGAGCGCCTGTACCCAGAGGACGGAGTG
CTGAAGGGCGACATCAAGATGGCTCTGCGGCTGAAGGACGGAGGACGCT
ACCTGGCCGATTTCAAGACCACATACAAGGCTAAGAAGCCCGTGCAGAT
GCCTGGAGCTTACAACGTGGACAGAAAGCTGGACATCACCTCCCACAAC
GAGGACTACACAGTGGTGGAGCAGTACGAGAGGTCTGAGGGCAGACACA
GCACCGGCGGAATGGATGAGCTGTACAAGTGAGATATCAAGCTTATCGA
TAATCAACCTCTGGATTACAAAATTTGTGAAAGATTGACTGGTATTCTT
AACTATGTTGCTCCTTTTACGCTATGTGGATACGCTGCTTTAATGCCTT
TGTATCATGCTATTGCTTCCCGTATGGCTTTCATTTTCTCCTCCTTGTA
TAAATCCTGGTTGCTGTCTCTTTATGAGGAGTTGTGGCCCGTTGTCAGG
CAACGTGGCGTGGTGTGCACTGTGTTTGCTGACGCAACCCCCACTGGTT
GGGGCATTGCCACCACCTGTCAGCTCCTTTCCGGGACTTTCGCTTTCCC
CCTCCCTATTGCCACGGCGGAACTCATCGCCGCCTGCCTTGCCCGCTGC
TGGACAGGGGCTCGGCTGTTGGGCACTGACAATTCCGTGGTGTTGTCGG
GGAAATCATCGTCCTTTCCTTGGCTGCTCGCCTGTGTTGCCACCTGGAT
TCTGCGCGGGACGTCCTTCTGCTACGTCCCTTCGGCCCTCAATCCAGCG
GACCTTCCTTCCCGCGGCCTGCTGCCGGCTCTGCGGCCTCTTCCGCGAC
TTCGCCTTCGCCCTCAGACGAGTCGGATCTCCCTTTGGGCCGCCTCCCC
GCAGATCTAACTTGTTTATTGCAGCTTATAATGGTTACAAATAAAGCAA
TAGCATCACAAATTTCACAAATAAAGCATTTTTTTCACTGCATTCTAGT
TGTGGTTTGTCCAAACTCATCAATGTATCTTAGAAGTTCCTATTCCGAA
GTTCCTATTCTTCAAATAGTATAGGAACTTCCCGAATGCATCTAGAGGA
TCCTCGAGCCCGTCGACCGATAAAAGTTTTGTTACTTTATAGAAGAAAT
TTTGAGTTTTTGtTTTTTTTTAATAAATAAATAAACATAAATAAATTGT
TTGTTGAATTTATTATTAGTATGTAAGTGTAAATATAATAAAACTTAAT
ATCTATTCAAATTAATAAATAAACCTCGATATACAGACCGATAAAACAC
ATGCGTCAATTTTACgCATGATTATCTTTAACGTACGTCACAATATGAT
TATCTTTCTAGGGTTAAAGGCCTTCGGTCGTTCGGCTGCGGCGAGCGGT
ATCAGCTCACTCAAAGGCGGTAATACGGTTATCCACAGAATCAGGGGAT
AACGCAGGAAAGAACATGTGAGCAAAAGGCCAGCAAAAGGCCAGGAACC
GTAAAAAGGCCGCGTTGCTGGCGTTTTTCCATAGGCTCCGCCCCCCTGA
CGAGCATCACAAAAATCGACGCTCAAGTCAGAGGTGGCGAAACCCGACA
GGACTATAAAGATACCAGGCGTTTCCCCCTGGAAGCTCCCTCGTGCGCT
CTCCTGTTCCGACCCTGCCGCTTACCGGATACCTGTCCGCCTTTCTCCC
TTCGGGAAGCGTGGCGCTTTCTCATAGCTCACGCTGTAGGTATCTCAGT
TCGGTGTAGGTCGTTCGCTCCAAGCTGGGCTGTGTGCACGAACCCCCCG
TTCAGCCCGACCGCTGCGCCTTATCCGGTAACTATCGTCTTGAGTCCAA
CCCGGTAAGACACGACTTATCGCCACTGGCAGCAGCCACTGGTAACAGG
ATTAGCAGAGCGAGGTATGTAGGCGGTGCTACAGAGTTCTTGAAGTGGT
GGCCTAACTACGGCTACACTAGAAGAACAGTATTTGGTATCTGCGCTCT
GCTGAAGCCAGTTACCTTCGGAAAAAGAGTTGGTAGCTCTTGATCCGGC
AAACAAACCACCGCTGGTAGCGGTGGTTTTTTTGTTTGCAAGCAGCAGA
TTACGCGCAGAAAAAAAGGATCTCAAGAAGATCCTTTGATCTTTTCTAC
GGGGTCTGACGCTCAGTGGAACGAAAACTCACGTTAAGGGATTTTGGTC
ATGAGATTATCAAAAAGGATCTTCACCTAGATCCTTTTAAATTAAAAAT
GAAGTTTT

TABLE 2
Primers for amplifying 9 transition
vectors in the construction of
91-guide transgene

| | | SEQ |
|---|---|---|
| Primer | | ID |
| name | Primer sequence | NO: |
| 1-F | GATATCCACTTGAGACCACGGTTATCCACA | 7 |
| | GAATC | |
| 1-R | GATATCAGGATGAGACCCACCGTCATCACCG | 8 |
| 2-F | GGTGATGACGGTGGGTCTCACTGGGATATC | 9 |
| 2-R | GATATCTCCTTGAGACCACGGTTATCCACA | 10 |
| 3-F | GATATCCATCTGAGACCCACCGTCATCACC | 11 |
| 3-R | GATATCCTGGTGAGACCACGGTTATCCACA | 12 |
| 4-F | GATATCCCTCTGAGACCCACCGTCATCACC | 13 |
| 4-R | GATATCGATGTGAGACCACGGTTATCCACA | 14 |
| 5-F | GATATCCAGATGAGACCCACCGTCATCACC | 15 |
| 5-R | GATATCGAGGTGAGACCACGGTTATCCACA | 16 |
| 6-F | GATATCACCATGAGACCCACCGTCATCACC | 17 |
| 6-R | GATATCTCTGTGAGACCACGGTTATCCACA | 18 |
| 7-F | GATATCCGCCTGAGACCCACCGTCATCACC | 19 |
| 7-R | GATATCTGGTTGAGACCACGGTTATCCACA | 20 |
| 8-F | GATATCCGAATGAGACCCACCGTCATCACC | 21 |
| 8-R | GATATCGGCGTGAGACCACGGTTATCCACA | 22 |
| 9-F | GATATCCAGTTGAGACCCACCGTCATCACC | 23 |
| 9-R | GATATCTTCGTGAGACCACGGTTATCCACA | 24 |

Although specific embodiments of the present disclosure have been described above, it should be understood by those skilled in the art that these embodiments are merely illustrative, and various changes or modifications can be made to these embodiments without departing from the principle and essence of the present disclosure. Accordingly, the scope of protection of the present disclosure is defined by the appended claims.

1. A LoxP nucleic acid pair comprising a TATA-Lox71 sequence and a TATA-LoxTC9 sequence;
wherein the TATA-Lox71 sequence and the TATA-LoxTC9 sequence are shown in SEQ ID NO: 1 and SEQ ID NO: 2, respectively.

2. A Cre-LoxP recombination system comprising a Cre enzyme and the LoxP nucleic acid pair according to claim 1; the LoxP nucleic acid pair can undergo only one round of Cre enzyme-mediated recombination.

3. A nucleic acid construct encoding a Cre-Lox recombination system and a CRISPR gene editing system, wherein the nucleic acid construct comprises a U6 promoter, sgRNA expression elements in tandem, and an inverted terminal repeat sequence of a transposon for inserting the nucleic acid construct into the genome of a target cell;
wherein the U6 promoter comprises a TATA-Lox71 sequence;
the nucleotide sequence of the TATA-Lox71 sequence and the nucleotide sequence of the U6 promoter are shown in SEQ ID NO: 1 and SEQ ID NO: 3, respectively;
the sgRNA expression element comprises, from the 5′ end to the 3′ end, an sgRNA targeting a target gene, a transcription terminator, and a TATA-LoxTC9 sequence; and
the nucleotide sequence of the TATA-LoxTC9 sequence is shown in SEQ ID NO: 2;
the LoxP nucleic acid pair according to claim 1 is capable of only one round of Cre enzyme-mediated recombination, resulting an induction of sgRNA expression; the sgRNA then recruits Cas proteins or derivatives thereof to perturb the target genes.

4. The nucleic acid construct according to claim 3, wherein the numbers of the sgRNA expression elements are more than 2, such as 60 to 150; or,
the transcription terminator is T6; or,
the nucleic acid construct is flanked by inverted terminal repeat sequence of a transposon; wherein the nucleotide sequence of the inverted terminal repeat sequence is shown in SEQ ID NO: 4.

5. The nucleic acid construct according to claim 3, wherein the sgRNA expression elements in tandem further comprise a stuffer before or after the first sgRNA expression element; and
the stuffer is a random inert sequence refractory to recombination.

6. The nucleic acid constructs according to claim 5, wherein the length of the stuffer sequence is 0.5 kb to 10 kb.

7. A recombinant expression vector comprising the LoxP nucleic acid pair according to claim 1.

8. A recombinant cell comprising the LoxP nucleic acid pair according to claim 1.

9. A method for preparing a single-gene perturbation animal line, wherein the method comprises:
using the nucleic acid construct according to claim 3, recombining TATA-Lox71 at the U6 promoter with TATA-LoxTC9 on the sgRNA expression elements using Cre enzyme, so that the sgRNA is randomly expressed in germ cells of an animal in vivo, and then
deriving an offspring line expressing the same sgRNA throughout the body via natural reproduction, followed by introducing a transgene expressing a Cas protein or derivatives thereof into the offspring line to obtain single-gene perturbation lines; or,
generating a mosaic animal with random gene perturbation, and then breeding single-gene perturbation lines.

10. (canceled)

11. The recombinant expression vector according to claim 7, wherein the recombinant expression vector further comprises a nucleotide sequence encoding a Cre enzyme or a Cas protein or derivatives thereof.

12. A recombinant expression vector comprising the Cre-LoxP recombination system according to claim 2.

13. The recombinant expression vector according to claim 12, wherein the recombinant expression vector further comprises a nucleotide sequence encoding a Cre enzyme or a Cas protein or derivatives thereof.

14. A recombinant expression vector comprising the nucleic acid construct according to claim 3.

15. The recombinant expression vector according to claim 14, wherein the recombinant expression vector further comprises a nucleotide sequence encoding a Cre enzyme or a Cas protein or derivatives thereof.

16. The recombinant cell according to claim 8, wherein the cell is derived from a mammalian cell line.

17. The recombinant cell according to claim 16, wherein the mammalian cell line is derived from mice, rats, or rabbits.

18. A recombinant cell comprising the nucleic acid construct according to claim 3.

19. A recombinant cell comprising the recombinant expression vector according to claim 7.

20. A method for preparing a single-gene perturbation animal line, wherein the method comprises:
using the nucleic acid construct according to claim 4, recombining TATA-Lox71 at the U6 promoter with TATA-LoxTC9 on the sgRNA expression elements using Cre enzyme, so that the sgRNA is randomly expressed in germ cells of an animal in vivo, and then
deriving an offspring line expressing the same sgRNA throughout the body via natural reproduction, followed by introducing a transgene expressing a Cas protein or derivatives thereof into the offspring line to obtain single-gene perturbation lines; or,
generating a mosaic animal with random gene perturbation, and then breeding single-gene perturbation lines.

21. A method for preparing a single-gene perturbation animal line, wherein the method comprises:
using the nucleic acid construct according to claim 5, recombining TATA-Lox71 at the U6 promoter with TATA-LoxTC9 on the sgRNA expression elements using Cre enzyme, so that the sgRNA is randomly expressed in germ cells of an animal in vivo, and then
deriving an offspring line expressing the same sgRNA throughout the body via natural reproduction, followed by introducing a transgene expressing a Cas protein or derivatives thereof into the offspring line to obtain single-gene perturbation lines; or,
generating a mosaic animal with random gene perturbation, and then breeding single-gene perturbation lines.


SEQUENCE LISTING (29 sequences)

SEQ ID NO: 1 - DNA, 34 bp, synthetic construct
  source 1..34: mol_type = other DNA
taccgttcgtatagtataaattatacgaagttat

SEQ ID NO: 2 - DNA, 34 bp, synthetic construct
  source 1..34: mol_type = other DNA
ataacttcgtatagtataaattattgcttcggta

SEQ ID NO: 3 - DNA, 312 bp, synthetic construct
  source 1..312: mol_type = other DNA
cgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaatttaccgttcgtatagtataaattatacgaagttataagccttgtttg

SEQ ID NO: 4 - DNA, 518 bp, synthetic construct
  source 1..518: mol_type = other DNA
attcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatagccgataaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaa

SEQ ID NO: 5 - DNA, 1840 bp, synthetic construct
  source 1..1840: mol_type = other DNA
agcaacgcggcctttttacggttcctggccttttgctggccttttgctcacatgttctttcctgcgttatcccctgattctgtggataaccgtggtctcagaaggatatccggttgagacccaccgtcatcaccgaaacgcgcgatgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctggctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacattattcagattgggcttgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgcc

SEQ ID NO: 6 - DNA, 4810 bp, synthetic construct
  source 1..4810: mol_type = other DNA
aaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttaggcctttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatagcttcgataccgtcggctcgagaatgcatctagaggatccccacaggtccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaatttaccgttcgtatagtataaattatacgaagttataagccttgtttgaatgtctcagaccatatggggtttaagagctatgctggaaacagcatagcaagtttaaataaggctagtccgttatcaacttgaaaaagtggcaccgagtcggtgctttttttgggaagttcctattccgaagttcctattcttcaaatagtataggaacttcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttcgaagttctgtatgagaccacgaaacaccggaattcgccaccatggtgagcaagggcgaggccgtgatcaaggagttcatgaggtttaaggtgcacatggagggcagcatgaacggccacgagttcgagatcgagggagagggagagggcagaccctacgagggcacccagacagctaagctgaaggtgaccaagggcggaccactgccctttagctgggacatcctgtcccctcagttcatgtacggcagcagggccttcatcaagcaccctgctgacatcccagattactacaagcagtctttcccagagggctttaagtgggagagagtgatgaacttcgaggacggcggagccgtgaccgtgacacaggacacctctctggaggatggaacactgatctacaaggtgaagctgcggggaacaaactttccccctgatggcccagtgatgcagaagaaaaccatgggatgggaggccagcacagagcgcctgtacccagaggacggagtgctgaagggcgacatcaagatggctctgcggctgaaggacggaggacgctacctggccgatttcaagaccacatacaaggctaagaagcccgtgcagatgcctggagcttacaacgtggacagaaagctggacatcacctcccacaacgaggactacacagtggtggagcagtacgagaggtctgagggcagacacagcaccggcggaatggatgagctgtacaagtgagatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgacttcgccttcgccctcagacgagtcggatctccctttgggccgcctccccgcagatctaacttgtttattgcagcttataatggttacaaataaagcaatagcatcacaaatttcacaaataaagcatttttttcactgcattctagttgtggtttgtccaaactcatcaatgtatcttagaagttcctattccgaagttcctattcttcaaatagtataggaacttcccgaatgcatctagaggatcctcgagcccgtcgaccgataaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaaggccttcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagtttt

SEQ ID NO: 7 - DNA, 35 bp, synthetic construct
  source 1..35: mol_type = other DNA
gatatccacttgagaccacggttatccacagaatc

SEQ ID NO: 8 - DNA, 31 bp, synthetic construct
  source 1..31: mol_type = other DNA
gatatcaggatgagacccaccgtcatcaccg

SEQ ID NO: 9 - DNA, 30 bp, synthetic construct
  source 1..30: mol_type = other DNA
ggtgatgacggtgggtctcactgggatatc

SEQ ID NO: 10 - DNA, 30 bp, synthetic construct
  source 1..30: mol_type = other DNA
gatatctccttgagaccacggttatccaca

SEQ ID NO: 11 - DNA, 30 bp, synthetic construct
  source 1..30: mol_type = other DNA
gatatccatctgagacccaccgtcatcacc

SEQ ID NO: 12 - DNA, 30 bp, synthetic construct
  source 1..30: mol_type = other DNA
gatatcctggtgagaccacggttatccaca

SEQ ID NO: 13 - DNA, 30 bp, synthetic construct
  source 1..30: mol_type = other DNA
gatatccctctgagacccaccgtcatcacc

SEQ ID NO: 14 - DNA, 30 bp, synthetic construct
  source 1..30: mol_type = other DNA
gatatcgatgtgagaccacggttatccaca

SEQ ID NO: 15 - DNA, 30 bp, synthetic construct
  source 1..30: mol_type = other DNA
gatatccagatgagacccaccgtcatcacc

SEQ ID NO: 16 - DNA, 30 bp, synthetic construct
  source 1..30: mol_type = other DNA
gatatcgaggtgagaccacggttatccaca

SEQ ID NO: 17 - DNA, 30 bp, synthetic construct
  source 1..30: mol_type = other DNA
gatatcaccatgagacccaccgtcatcacc

SEQ ID NO: 18 - DNA, 30 bp, synthetic construct
  source 1..30: mol_type = other DNA
gatatctctgtgagaccacggttatccaca

SEQ ID NO: 19 - DNA, 30 bp, synthetic construct
  source 1..30: mol_type = other DNA
gatatccgcctgagacccaccgtcatcacc

SEQ ID NO: 20 - DNA, 30 bp, synthetic construct
  source 1..30: mol_type = other DNA
gatatctggttgagaccacggttatccaca

SEQ ID NO: 21 - DNA, 30 bp, synthetic construct
  source 1..30: mol_type = other DNA
gatatccgaatgagacccaccgtcatcacc

SEQ ID NO: 22 - DNA, 30 bp, synthetic construct
  source 1..30: mol_type = other DNA
gatatcggcgtgagaccacggttatccaca

SEQ ID NO: 23 - DNA, 30 bp, synthetic construct
  source 1..30: mol_type = other DNA
gatatccagttgagacccaccgtcatcacc

SEQ ID NO: 24 - DNA, 30 bp, synthetic construct
  source 1..30: mol_type = other DNA
gatatcttcgtgagaccacggttatccaca

SEQ ID NO: 25 - DNA, 34 bp, synthetic construct
  source 1..34: mol_type = other DNA
ataacttcgtatagtataaattatacgaagttat

SEQ ID NO: 26 - DNA, 34 bp, synthetic construct
  source 1..34: mol_type = other DNA
ataacttcgtatagtataaattataccttgttat

SEQ ID NO: 27 - DNA, 34 bp, synthetic construct
  source 1..34: mol_type = other DNA
taccgttcgtatagtataaattataccttgttat

SEQ ID NO: 28 - DNA, 33 bp, synthetic construct
  source 1..33: mol_type = other DNA
taccgttcgtatagtataaattatgcttcggta

SEQ ID NO: 29 - DNA, 43 bp, synthetic construct
  source 1..43: mol_type = other DNA
ggtaaagccttgtttgggaaaactctcacctcaacggtttaag